(19) United States
(12) Patent Application Publication
(10) Pub. No.: US 2006/0281814 A1
(43) Pub. Date: Dec. 14, 2006

(54) Conjugated linolenic acids and methods for commerical preparation and purification

(76) Inventors: Paul Angers, Sainte Foy (CA); Frederic Destaillats, Belmont sur Lausanne (CH); Juan Miguel Garro Galvez, Sherbrooke (CA)

Correspondence Address: KNOBBE MARTENS OLSON & BEAR LLP, 2040 MAIN STREET, FOURTEENTH FLOOR, IRVINE, CA, 92614, US

(21) Appl. No.: 10/523,863

(22) PCT Filed: Aug. 6, 2003
(86) PCT No.: PCT/CA03/01183

§ 371 Date: Jan. 17, 2006

(30) Foreign Application Priority Data
Aug. 6, 2002 (CA) 2,396,840

(51) Int. Cl. A61K 31/202; C07C 51/347

(52) U.S. Cl. 514/560; 554/126

(57) ABSTRACT

A method for the preparation and purification of conjugated linolenic acids is described. The method comprises blending a mixture of vegetable oils and or fats including various concentrations of alpha or gamma and or both linolenic acids with a base. The method transforms approximately over two thirds of α-linolenic acid (9Z,12Z,15Z-octadecatrienoic acid) into 9Z,11E,15Z-octadecatrienoic acid and 9Z,13E,15Z-octadecatrienoic acid. The method also transforms gamma-linolenic acid (6Z,9Z,12Z-octadecatrienoic acid) into 6Z,8E,15Z-octadeccatrienoic acid and 6Z,10E,12Z-octadecatrienoic acid. In all cases, geometrical isomers and fully conjugated isomers are also produced.

### FIELD OF THE INVENTION

The present invention relates to a method for the preparation and purification of fatty acids which are homologues of conjugated linoleic acids, from materials rich in alpha or gamma linolenic acids. The method permits the transformation of approximately over two thirds of a-linolenic acid (9Z,12Z,15Z-octadecatrienoic acid) into 9Z,11E,15Z-octadecatrienoic acid and 9Z,13E,15Z-octadecatrienoic acid. Enrichment up to and over 40% is readily performed with urea crystallization. Moreover, the product can be produced in over 90% purity by simple preparative liquid chromatography. The reaction is unique in that the reaction produces the above mentioned conjugated trienoic acids with a high selectivity, in a short time period and in relatively mild conditions. The reaction also transforms gamma-linolenic acid (6Z,9Z,12Z-octadecatrienoic acid) into 6Z,8E,12Z-octadeccatrienoic acid and 6Z,10E,12Z-octadecatrienoic acid. In all cases, geometrical isomers and fully conjugated isomers are also produced.

### BACKGROUND OF THE INVENTION

Processes for the conjugation of the double bonds of polyunsaturated unconjugated fatty acids have found their main application in the field of paints and varnishes. Oils comprised of triglycerides of conjugated fatty acids are known as drying oils. Drying oils have value because of their ability to polymerize or “dry” after they have been applied to a surface to form tough, adherent and abrasion resistant films. Tung oil is an example of a naturally occurring oil containing significant levels of fully conjugated fatty acids. Because tung oil is expensive for many industrial applications, research was directed towards finding substitutes.

In the 1930's, it was found that conjugated fatty acids were present in oil products subjected to prolonged saponification, as originally described by Moore (J. Biochem., 31: 142 (1937)). This finding led to the development of several alkali isomerization processes for the production of conjugated fatty acids from various sources of polyunsaturated fatty acids.

The positioning of the double bonds in the hydrocarbon chain is typically not in a conjugated, i.e., alternating double bond-single bond-double bond, manner. For example, α-linolenic acid is an eighteen carbon acid with three double bonds (18:3) at carbons 9, 12 and 15 in which all three double bonds have the cis configuration, i.e., 9Z,12Z,15Z-C18:3 acid. α-Linolenic acid is 6Z,9Z,12Z-C18:3 acid and linoleic acid is 9Z,12Z-C18:2 acid (see TABLE 1).

TABLE 1

| N^{o} | Fatty Acid | Trivial Name | Structure |
|---|---|---|---|
| 1 | 9Z, 12Z, 15Z-C18:3 | α-Linolenic Acid | 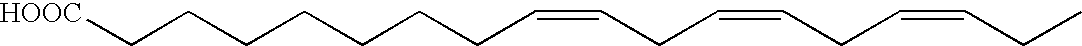 |
| 2 | 6Z, 9Z, 12Z-C18:3 | γ-Linolenic Acid | 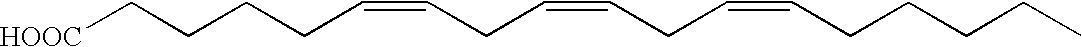 |
| 3 | 9Z, 12Z-C18:2 | Linoleic Acid | 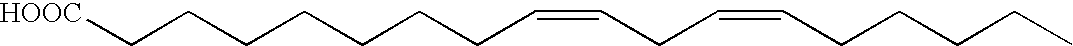 |

Migration of double bonds (e.g., leading to conjugation) gives rise to many positional and geometric (i.e., cis-trans) isomers.

Conjugated double bonds means two or more double bonds which alternate with single bonds as in 1,3-butadiene. The hydrogen atoms are on the same side of the molecule in the case of cis-structure. The hydrogen atoms are on opposite sides of the molecule in the case of trans-structure.

Conjugated linoleic acid (CLA) is a general term used to name positional and geometric isomers of linoleic acid. Linoleic acid is a straight chain carboxylic acid having double bonds between the carbons 9 and 10, and between carbons 12 and 13. For example, one CLA positional isomer has double bonds between carbons 9 and 10 and carbons 11 and 12 (i.e., 9Z,11E-C18:2 acid); another has double bonds between carbons 10 and 11 and carbons 12 and 13 (i.e., 10E,12Z-C18:2 acid), each with several possible cis-and trans-isomers (see Table 2).

TABLE 2

| N^{o} | Fatty Acid | Trivial Name | Structure |
|---|---|---|---|
| 1 | 9Z, 11E-C18:2 | Rumenic Acid | 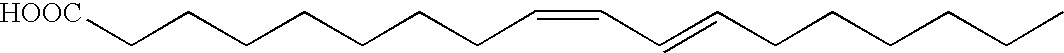 |
| 2 | 10E, 12Z-C18:2 | none | 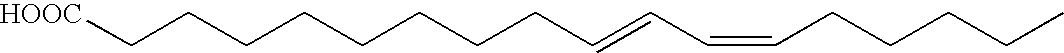 |

The 9Z,11E-C18:2 isomer has been shown to be the first intermediate produced in the biohydrogenation process of linoleic acid by the anaerobic rumen bacterium Butyrvibrio fibrisolvens. This reaction is catalyzed by the enzyme Δ11 isomerase which converts the cis-12 double bond of linoleic acid into a trans-11 double bond (C. R. Kepler et al., 241, J. Biol. Chem. (1966) 1350). It has also been found that the normal intestinal flora of rats can also convert linoleic acid to the 9Z,11E-C18:2 acid isomer. The reaction does not, however, take place in animals lacking the required bacteria. Therefore, CLA is largely a product of microbial metabolism in the digestive tract of primarily ruminants, but to a lesser extent in other mammals and birds.

The free, naturally occurring conjugated linoleic acids (CLA) have been previously isolated from fried meats and described as anticarcinogens by Y. L Ha, N K. Grimm and M. W. Pariza (Carcinogenesis, Vol. 8, No. 12, pp. 1881-1887 (1987)). Since then, they have been found in some processed cheese products (Y. L. Ha, N. K. Grimm and M. W. Pariza, J. Agric. Food Chem., Vol. 37, No. 1, pp. 75-81 (1987)). Cook et al. (U.S. Pat. No. 5,554,646) disclose animal feeds containing CLA, or its non-toxic derivatives, e.g., such as sodium and potassium salts of CLA, as an additive in combination with conventional animal feeds or human foods. CLA makes for leaner animal mass.

The biological activity associated with CLAs is diverse and complex (Pariza et al., Prog. Lipid Research., Vol 40, pp. 283-298).

Anti-carcinogenic properties have been well documented, as well as stimulation of the immune system. Administration of CLA inhibits rat mammary tumorogenesis, as demonstrated by Ha et al., (Cancer Res., 52:2035-s (1992)). Ha et al., (Cancer Res., 50:1097 (1990)), reported similar results in a mouse forestomach neoplasia model. CLA has also been identified as a strong cytotoxic agent against target human melanoma, colorectal and breast cancer cells in vitro. A recent major review article confirms the conclusions drawn from individual studies (Ip, Am. J. Clin. Nutr. 66(6):1523s (1997)). In in vitro tests, CLAs were tested for their effectiveness against the growth of malignant human melanomas, colon and breast cancer cells. In the culture media, there was a significant reduction in the growth of cancer cells treated with CLAs by comparison with control cultures. The mechanism by which CLAs exert anticarcinogenic activity is unknown. In addition, CLAs have a strong antioxidative effect so that, for example, peroxidation of lipids can be inhibited (Atherosclerosis 108, 19-25 (1994)). U.S. Pat. 5,914,346 discloses the use of CLAs to enhance natural killer lymphocyte function. U.S. Pat. No. 5,430,066 describes the effect of CLAs in preventing weight loss and anorexia by immune system stimulation.

Although the mechanisms of CLA action are still obscure, there is evidence that some component(s) of the immune system may be involved, at least in vivo. U.S. Pat. No. 5,585,400 (Cook, et al.), discloses a method for attenuating allergic reactions in animals mediated by type I or IgE hypersensitivity, by administering a diet containing CLA. CLA in concentrations of about 0.1 to about 1.0 percent was also shown to be an effective adjuvant in preserving white blood cells. U.S. Pat. No. 5,674,901 (Cook, et al.), teaches that oral or parenteral administration of CLA in either free acid or salt form resulted in an elevation in CD-4 and CD-8 lymphocyte subpopulations associated with cell mediated immunity. Adverse effects arising from pretreatment with exogenous tumor necrosis factor could be alleviated indirectly by elevation or maintenance of levels of CD-4 and CD-8 cells in animals to which CLA was administered.

CLAs have also been found to exert a profound generalized effect on body composition, in particular, upon redirecting the partitioning of fat and lean tissue mass. U.S. Pat. Nos. 5,554,646 and 6,020,378 teach the use of CLAs for reducing body fat and increasing lean body mass. U.S. Pat. No. 5,814,663 teaches the use of CLAs to maintain an existing level of body fat or body weight in humans. U.S. Pat. No. 6,034,132 discloses the use of CLAs to reduce body weight and treat obesity in humans. CLAs are also disclosed in U.S. Pat. No. 5,804,210 to maintain or enhance bone mineral content. EP 0 579 901 B relates to the use of CLA for avoiding loss of weight or for reducing increases in weight or anorexia caused by immunostimulation in humans or animals. U.S. Pat. No. 5,430,066 (Cook, et al.), teaches the effect of CLA in preventing weight loss and anorexia by immune stimulation.

CLA has been found to be an in vitro antioxidant, and in cells, it protects membranes from oxidative attack. In relation to other important dietary antioxidants, it quenches singlet oxygen less effectively than β-carotene but more effectively than α-tocopherol. It appears to act as a chain terminating antioxidant by chain-propagating free radicals (U.S. Pat. No. 6,316,645).

Skin is subject to deterioration through dermatological disorders, environmental abuse (wind, air conditioning, central heating) or through the normal aging process (chronoaging) which may be accelerated by exposure of skin to sun (photoaging). In recent years the demand for cosmetic compositions and cosmetic methods for improving the appearance and condition of skin has grown enormously. WO 95/13806 teaches the use of a composition comprising zinc salts of 68% (unconjugated) linoleic acid and 10% conjugated isomers of linoleic acid for use in treating skin disorders.

Apart from potential therapeutic and pharmacological applications of CLA as set forth above, there has been much excitement regarding the use of CLA as a dietary supplement. CLA has been found to exert a profound generalized effect on body composition, in particular redirecting the partitioning of fat and lean tissue mass. U.S. Pat. No. 5,554,646 (Cook, et al.), teaches a method utilizing CLA as a dietary supplement in which pigs, mice, and humans were fed diets containing 0.5% CLA. In each species, a significant drop in fat content was observed with a concomitant increase in protein mass. It is interesting that in these animals, increasing the fatty acid content of the diet by the addition of CLA resulted in no increase in body weight, but was associated with a redistribution of fat and lean tissue mass within the body. Another dietary phenomenon of interest is the effect of CLA supplementation on feed conversion. U.S. Pat. No. 5,428,072 (Cook, et al.), discloses data showing that the incorporation of CLA into animal feed (birds and mammals) increased the efficiency of feed conversion leading to greater weight gain in the CLA supplemented birds and mammals. The potential beneficial effects of CLA supplementation for food animal growers is apparent.

Another important source of interest in CLA, and one which underscores its early commercial potential, is that it is naturally occurring in foods and feeds consumed by humans and animals alike. In particular, CLA is abundant in products from ruminants. For example, several studies have been conducted in which CLA has been surveyed in various dairy products. Aneja, et al., (J. Dairy Sci., 43: 231 [1990]) observed that processing of milk into yogurt resulted in a concentration of CLA. Shanta, et al. (Food Chem., 47: 257 [1993]) showed that a combined increase in processing temperature and addition of whey increased CLA concentration during preparation of processed cheese. In a separate study, Shanta, et al., (J. Food Sci., 60: 695 [1995]) reported that while processing and storage conditions did not appreciably reduce CLA concentrations, they did not observe any increases. In fact, several studies have indicated that seasonal or interanimal variation can account for as much as three fold differences in the CLA content of cows milk (Parodi, et al., J. Dairy Sci., 60: 1550 [1977]). Also, dietary factors have been implicated in CLA content variation (Chin, et al., J. Food Comp. Anal., 5: 185 [1992]). Because of this variation in CLA content in natural sources, ingestion of prescribed amounts of various foods will not guarantee that the individual or animal will receive the optimum doses to ensure achieving the desired nutritive effect.

Economical conjugated fatty acid production in commercial quantities for use in domestic food animal feeds is a desirable objective in light of the nutritional benefits realized on a laboratory scale. Preferably, the conjugated fatty acid is produced directly from a source of raw vegetable oil and not from expensive purified linoleic acid. Further, the process must avoid cost generating superfluous steps, and yet result in a safe and wholesome product palatable to animals.

Useful methodologies for the preparation of conjugated linoleic acid (CLA) have been recently reviewed by Adlof (In:Yurawecz et al. (Ed), Advances in Conjugated Linoleic Acid Research, volume 1, AOCS Press, Champaign, Ill., pp 21-38 [1999]).

The usual methodology for conjugation of polyunsaturated fatty acids is alkali-catalyzed isomerization. This reaction may be performed using different bases such as hydroxides or alkoxides in solution in appropriate alcoholic reagents. This reaction was developed in the 1950's for the spectrophotometric estimation of polyunsaturated fatty acids in fats and oils [AOCS official method Cd 7-58; JAOCS 30:352 (1953)].

In alkali isomerization the fatty acids are exposed to heat, pressure and a metal hydroxide or oxide in nonaqueous or aqueous environments, resulting in the formation of conjugated isomers. Other methods have been described which utilize metal catalysts, but which are not as efficient for the operation of conjugated double bonds. It was found that isomerization could be more rapidly achieved in the presence of higher molecular weight solvents. Kass, et al., (J. Am. Chem. Soc., 61: 4829 (1939)) and U.S. Pat. No. 2,487,890 teach that the replacement of ethanol with ethylene glycol resulted in an increase in conjugation in less time. U.S. Pat. No. 2,350,583 and British Patent 558,881 teach conjugation by reacting fatty acid soaps of an oil with an excess of aqueous alkali at 200-230° C. and increased pressure.

Dehydration of methyl ricinoleate (methyl 12-hydroxy-cis-9-octadecenoate) (Gunstone and Said, Chem. Phys. Lipids 7, 121 [1971]; Berdeaux et al., JAOCS 74, 1011 [1997]) yields the 9Z,11E-C18:2 isomer as a major product. U.S. Pat. No. 5,898,074 teaches a synthetic process for producing this fatty acid at room temperature in high yield. The tosylate or the mesylate of the methyl ester of ricinoleic acid is formed with tosyl chloride or mesyl chloride in a pyridine solvent or in acetonitrile and triethyl amine. The obtained tosylate or mesylate was reacted with diazabicycloundecene in a polar, non-hydroxylic solvent such as acetonitrile to form the preferred isomer 9Z,11E-18:2 methyl ester in high yield. U.S. Pat. No. 6,160,141 discloses a synthetic process for producing conjugated eicosanoid fatty acid from methyl lesquerolate (methyl 14-hydroxy-cis-11-octadecenoate) at room temperature in high yield using the same principle.

Among the processes known to effect isomerization, without utilizing an aqueous alkali system, is a nickel-carbon catalytic method, as described by Radlove, et al., Ind. Eng. Chem.38: 997 (1946). A variation of this method utilizes platinum or palladium-carbon as catalysts. Conjugated acids may also be obtained from a-hydroxy allylic unsaturated fatty adds using acid catalyzed reduction (Yurawecz et al., JAOCS 70, 1093 [1993]) as well as by the partial hydrogenation of conjugated acetylenic acid such as santalbic (11E-octadec-9-ynoic) acid using Lindlar's catalyst but the methods are limited by natural sources of such fatty acids. Another approach using strong organic bases such as butyllithium has been applied to both the conjugation of linoleic acid and the partial and full conjugation of alpha-linolenic acid (U.S. Pat. No. 6,316,645).

Natural fully conjugated linolenic acids have been found at high content levels in some seed oils (Hopkins, In:Gunstone, F. D. (Ed), Topics in Lipid Chemistry, volume 3, ELEK Science, London, pp 37-87 [1972]). For example, Takagi and Itabashi (Lipids 16, 546 [1981]) reported calendic acid (8E,10E,12Z-C18:3 acid, 62.2%) in pot marigold seed oil, punicic acid (9Z,11E,13Z-C18:3 acid, 83.0%) in pomegranate seed oil; α-eleostearic acid (9Z,11E,13E-C18:3 acid) in tung (67.7%) and bitter gourd (56.2%) seed oils; and catalpic acid (9E,11E,13Z-C18:3 acid, 42.3%) in catalpa seed oil, respectively.

An octadecatrienoic acid isomer whose structure has been tentatively defined as 9Z,11E,15Z-C18:3 acid, is believed to be the first intermediate in the biohydrogenation process of α-linolenic acid by the anaerobic rumen bacterium Butyrvibrio fibrisolvens (C. R. Kepler and S. B. Tove 242 J. Biol. Chem. (1967) 5686).

There thus remains a need to develop a method for the preparation and purification of new conjugated linolenic acids.

The present invention seeks to meet these and other needs.

The present invention refers to a number of documents, the content of which is herein incorporated by reference in their entirety.

### SUMMARY OF THE INVENTION

The present invention relates to a method for the preparation and purification of fatty acids which are homologues of conjugated linoleic acids, from natural and/or synthetic materials richin alpha or gamma linolenic acids or both. In a preferred embodiment, the method transforms approximately over two thirds of alpha linolenic acid (9Z,12Z,15Z-C18:3 acid), from a natural source such as linseed oil, into 9Z,11E,15Z and 9Z,13E,15Z C18:3 acids, producing a mixture comprising approximately 30% of the conjugated linolenic acids. In a further embodiment, enrichment up to and over 40% is readily performed with urea crystallization. Moreover, the product is obtained in over 90% purity by simple preparative liquid chromatography. The products obtained include free fatty acids, and derivatives thereof, including, but not limited to esters, amides, salts as well as fatty alcohols The method of the present invention produces the above mentioned conjugated trienoic acid with a high selectivity, in a short time period and under relatively mild conditions

The present invention further relates to a method for preparing conjugated linolenic acids comprising the steps of:
(a) blending a or a mixture of vegetable oils and/or fats including various concentrations of alpha or gamma and or both linolenic acids with a base to produce a reaction mixture;
(b) recovering said conjugated linolenic acids from the reaction mixture, and
(c) subjecting the reaction mixture to urea complexation or liquid chromatography.

Further scope and applicability will become apparent from the detailed description given hereinafter. It should be understood however, that this detailed descripton, while indicating preferred embodiments of the invention, is given by way of illustration only, since various changes and modifications within the spirit and scope of the invention will become apparent to those skilled in the art.

### BRIEF DESCRIPTION OF FIGURES

FIG. 1 shows mass spectra of products resulting from the isomerization process of alpha-linolenic acid (9Z,12Z,15Z-C18:3 acid), as 4,4-dimethyloxazoline derivatives: A, 9Z,11E,15Z and 9Z,13E,15Z-C18:3; B, 9,11,13-C18:3, C, 10E,12Z,14E-C18:3 and D, 11,13-CCLA (9-(Spropylcyclohexa-2,4-dienyl)-nonanoic acid);

FIG. 2 shows the mass sprectrum of the MTAD adducts of cis-9, trans-11, cis-5 18:3 (A) and cis-9, trans-13, cis-15 18:3 (B) acid, methyl esters;

FIG. 3 shows the thermal mechanism leading to the formation of 11,13-CCLA [9-(6propyl-cyclohexa-2,4-dienyl)-nonanoic acid (FIG. 1-D)] from 10E,1Z,14E-C18:3 acid;

FIG. 4 illustrates gas liquid chromatograms of fatty acid methyl esters obtained after methylation of linseed oil (A), conjugated linseed oil (B), liquid phase from urea crystallization (C), reversed-phase liquid chromatography fraction containing about 97% of a mixture of 9Z,11E,15Z and 9Z,13E,15Z-C18:3 acids (D), argentation liquid chromatography fraction containing about 99+% of a mixture of 9Z,11E,15Z and 9Z,13E,15Z-C18:3 acids (E);

FIG. 5 illustrates the gas liquid chromatogram of the fatty acid methyl esters obtained after methylation of partially conjugated evening primrose oil.

### DETAILED DESCRIPTION OF THE INVENTION

The oils and fats, alone or as mixtures, containing alpha-linolenic acid may include but are not limited to amebia, basil, candelnut, flax (linseed), linola, gold of pleasure, hemp, mustard, perilla, soybean, canola, walnut, chia, crambe, echium, hop, kiwi, pumpkin, black currant and purslane seed oils, or any other oil, wax, ester or amide that is rich in linolenic acid.

The oils and fats, alone or as mixtures, containing gamma-linolenic acid may include but are not limited to borage, evening primrose and black currant seed oils, or any other oil, wax, ester or amide that is rich in linolenic add.

The disclosed method converts double bonds of α- and γ-linolenic acid isomers into partly and/or fully conjugated systems as well as into cyclic fatty acid isomers. The process, which can be performed both in batch and continuous modes, involves blending one or a mixture of vegetable oils with various concentrations of alpha or gamma linolenic acids or both or partial glycerides of such oils, or partially purified or concentrated isomers with about 0.5 to about 10 moles of base such as sodium hydroxide, sodium alkoxylate, sodium metal, potassium hydroxide, potassium alkoxylate, potassium metal, and strong base resins. The reaction proceeds at temperatures from about 20° C. to about 280° C. in a solvent, selected from commercial polyols such as propylene glycol, glycerol and ethylene glycol, for periods ranging from about 30 seconds to about 18 hours, depending on the base and/or the temperature and/or solvent, and/or substrate and/or a desired expected conversion rate. After cooling, if required, to about 20-80° C., acid is added to the reaction mixture to neutralize the soaps and/or remaining base in the reactor. It is preferred to bring the pH of the contents of the reactor to a value of about 4 or less through the addition of either a mineral or organic acid. Acids that may be used include, but are not limited to, hydrochloric acid, sulfuric acid, phosphoric acid and citric acid. The solvent phase (polyol+water) is withdrawn and the remaining fatty acid rich phase can be washed with water and/or saline solutions of variable concentrations such as sodium chloride (5% w/w) to remove traces of acids used for acidification of the reaction mixture. Remaining water can be removed by usual means (ie. centrifugation, vacuum, distillation or drying agents). As described in Example 1, the concentration of 9Z,11E,15Z and 9Z,13E,15Z-C18:3 acid in the product is approximately 33/. This product, as such or converted into derivatives, can be used in nutrition, cosmetic, nutraceutical, biological and/or animal feed applications.

The isomer composition of the formed fatty acid was determined using gas-liquid chromatography coupled with a mass-spectrometer (GC-MS) of their corresponding 4,4-dimethyloxazoline (DMOX) derivatives. The use of derivatives is a necessary step prior to the structural determination of fatty acids by GC-MS because the mass spectra of fatty acid methyl esters, the usual derivatives for gas-liquid chromatography analysis, are devoid of sufficient information for the identification of structural isomers. This is mainly due to the high sensitivity of the carboxyl group to fragmentation and to double bond migration (Christie, W. W., Gas Chromatography-Mass Spectrometry Methods for Structural Analysis of Fatty Acids, Lipids 33:343-353 (1998)). However, stabilization of the carboxyl group by the formation of a derivative containing a nitrogen atom results in mass spectra that allows for the structural determination of most fatty acids. Indeed, these fatty acid derivatives provide diagnostic fragments that allow accurate structure determination. The derivatives were submitted to GC-MS using a Hewlett Packard 5890 Series II plus gas chromatograph attached to an Agilent model 5973N MS Engine. The latter was used in the electron impact mode at 70 eV with a source temperature of 230° C. For the DMOX derivatives, an open tubular capillary column coated with BPX-70 (60 m.times.0.25 mm, 0.25 μm film; SGE, Melbourne, Australia) was used. After holding the temperature at 60° C. for 1 minute, the oven temperature was increased by temperature-programming at 20° C./minute to 170° C. where it was held for 30 minutes, then at 5° C./minute to 210° C. where it was held for 30 minutes. Helium was the carrier gas at a constant flow-rate of 1 mL/minute, maintained by electronic pressure control.

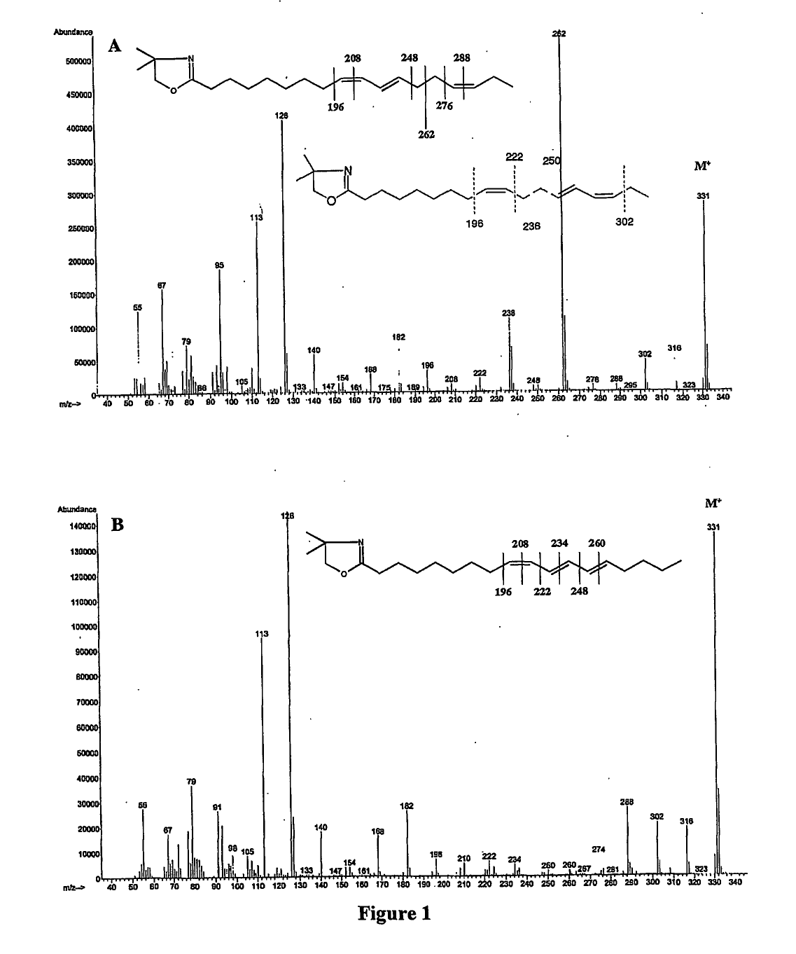

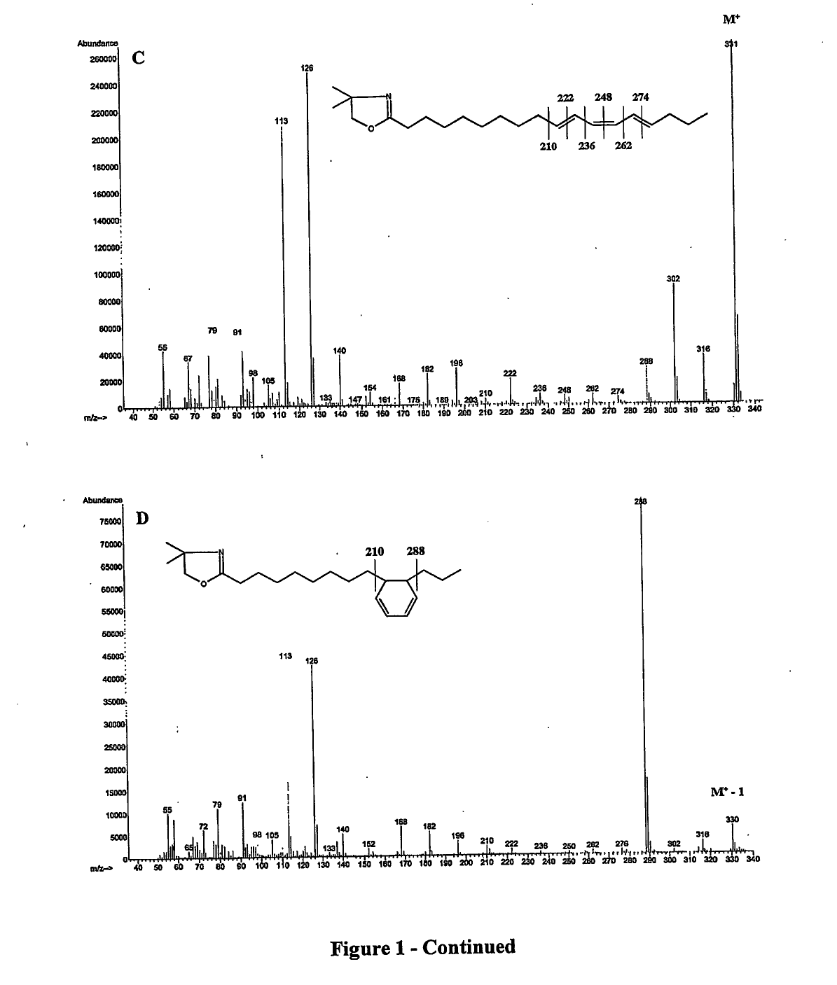

The mass spectrum of the conjugated products of 9Z,12Z,15Z-C18:3 acid, obtained by conjugation of linseed oil, are presented in FIG. 1.

The structural formula and mass spectrum of the DMOX derivatives of the 9Z,11E,15Z-C18:3 acid are illustrated in FIG. 1A. DMOX has a molecular ion at m/z=331, confirming the octadecatrienoic acid structure. The ion at m/z=262 confirms the location of the 11,15-double bond system (by extrapolation from the known 5,9-isomer (Berdeaux and Wolff, J. Am. Oil Chem. Soc., 73: 1323-1326 (1996)), similarly, the molecular ion at m/z=236 confirms the location of the 9,13-double bond system, and gaps of 12 a.m.u. between m/z=208 and 196, and 288 and 276 verify the location of double bonds in positions 9 and 15, respectively. Mass spectrometry does not however confirm the geometry of the double bonds, but they have been determined according to Nichols et al. (J. Am. Chem. Soc, 73:247-252 (1951)) based on the Ingold theory on the prototropic shift mechanism (Ingold, J. Chem. Soc, 1477 (1926)).

The structural formula and mass spectrum of the DMOX derivatives of the 9,11,13-C18:3 acid are illustrated in FIG. 1B. DMOX has a molecular ion at m/z=331, confirming the octadecatrienoic acid structure. Gaps of 12 a.m.u. between m/z=208 and 196, and 222 and 234, and 248 and 260 verify the location of the double bonds in positions 9, 11 and 13, respectively. Four different minor isomers of 9,11,13-C18:3 are present in the reaction products. The most abundant is the 9Z,11Z,13E-C18:3 acid isomer which is known as a-eleostearic add.

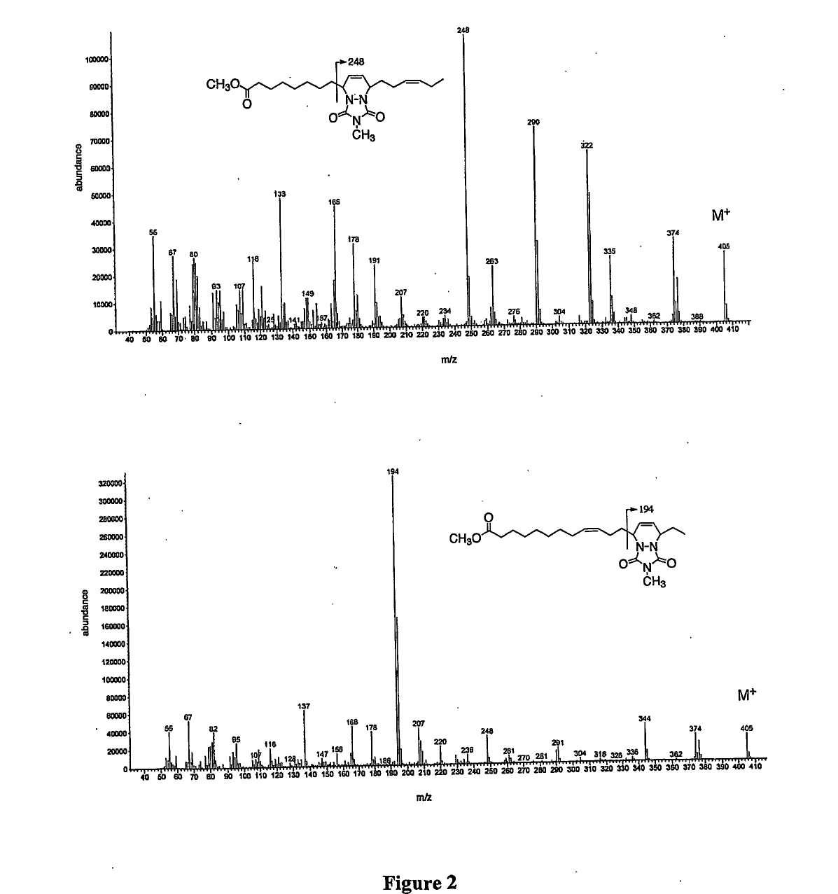

The mass spectra of the MTAD adducts of cis-9,trans-11 ,cis-15 18:3 (A) and cis-9,trans-13,cis-15 18:3 (B) acid methyl esters and presented in FIG. 2.

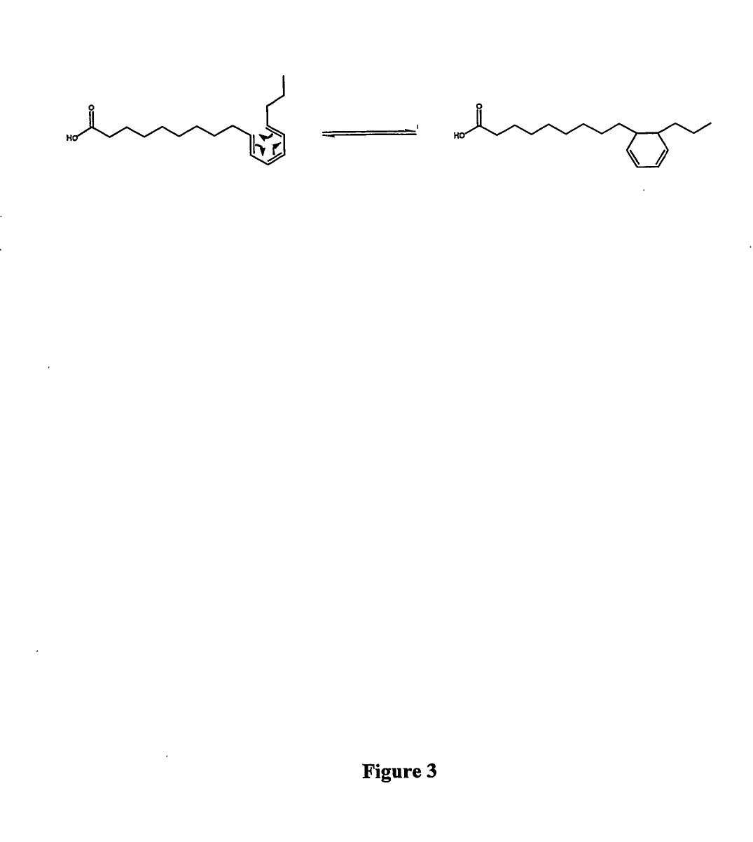

The structural formula and mass spectrum of the DMOX derivatives of the 10E,12Z,14E-C18:3 acid are illustrated in FIG. 1C. DMOX has a molecular ion at m/z=331, confirming the octadecatrienoic acid structure. Gaps of 12 a.m.u. between m/z=210 and 222, and 236 and 248, and 262 and 274 verify the location of the double bonds in positions 10, 12 and 14, respectively. The geometry of the double bonds, has been determined according to Nichols et al. (J. Am. Chem. Soc, 73:247-252 (1951)) based on the Ingold theory on the prototropic shift mechanism (Ingold, J. Chem. Soc, 1477 (1926)). The 10E,12Z,14E-C18:3 acid isomer is prone to cyclization, thus forming the cyclohexadienyl compound (9-(6-propyl-cyclohexa-2,4dienyl)-nonanoic acid)) by an electrocyclization process presented in FIG. 3.

The structural formula and mass spectrum of the DMOX derivatives of the 11,13-CCLA (9-(6-propyl-cyclohexa-2,4-dienyl)-nonanoic acid) are illustrated in FIG. 1 D. DMOX has a molecular ion at m/z=330−1, confirming the occurrence of a highly stabilized conjugated ion fragment (radical in carbon 10 or 15, stabilized by resonance effect). A distinctive ion at m/z=288 is characteristic of alpha cleavage occurring in cyclic fatty acids (Sébédio et al. J. Am. Oil Chem. Soc., 64: 1324-1333 (1987)). The gap of 78 atomic mass units (a.m.u.) between m/z=288 and 210 is that expected for the cyclohexadienyl group having a conjugated double bond system in positions 11 and 13.

The reaction progress was determined by gas-liquid chromatography under appropriate condition as presented in Example 1.

An increase in the concentration of, for example the 9Z,11E,15Z and 9Z,13E,15Z-C18:3 acids, can be achieved using different methods, alone or in combination. One method makes use of urea complexation. A urea solution is prepared at a temperature ranging from about 20 to 90° C. in different solvents or mixtures thereof, selected from water, and/or alcohols. Complexation is performed at the same temperature by addition of the product in a molar ratio of about 0.5 to 8, and cooling to a temperature range of about 30° C. to about −30° C., as required. A mixture of the above mentioned 9Z,11E,15Z and 9Z,13E,15Z-C18:3 acids is isolated in higher concentration following treatment of the liquid phase, obtained after separation from the solid phase using conventional means such as filtration or centrifugation. Decomplexation is then carried out by the addition of either a diluted organic or mineral acid. Acids that may be used include, but are not limited to, hydrochloric acid, sulfuric acid, phosphoric acid and citric acid. The product is obtained by decantation or liquid-liquid extraction with an organic solvent such as but not limited to hexane, heptane, petroleum ether and ligroin. If required, the organic solvent is eliminated (i.e. evaporation or distillation). A preferred description of the present embodiment is described in Example 2.

Another method for raising the level of, for example the 9Z,11E,15Z and 9Z,13E,15Z -C18:3 acids, either as free acids or derivatives (i.e. methyl, ethyl, isopropyl, butyl, phenyl) comprises the use of liquid chromatography using various convenient stationary phases. One particular chromatographic method is reversed phase liquid chromatography (i.e. ODS) for which eluents may include but are not limited to water, acetonitrile, acetone, methanol, tetrahydrofuran, methyltertbutyl ether, and combinations thereof. A detailed description of this method is provided in Example 3.

Argentation liquid chromatography may be used to isolate specific isomers from a complex mixture of fatty acid esters or free fatty acids. A detailed description of this methodology applied to a mixture of 9Z,11E,15Z and 9Z,13E,15Z-C18:3 acid isomers is described in Example 4.

Still another method for raising the concentration level of, for example, a mixture of 9Z,11E,15Z and 9Z,13E,15Z-C18:3 acids, either as free acids or derivatives (i.e. methyl, ethyl, isopropyl, butyl, phenyl) is crystallization, either in a solvent such as, but not limited to, acetone, methanol, pentane, or in mixtures therefor, or in the absence of a solvent (i.e. dry fractionation). A detailed cooling program is required in order to obtain a more concentrated product. One particular case is that of further crystallization of urea complexes of fatty acids.

Experimental

In the experimental disclosure which follows, the following abbreviations apply: kg (kilograms); g (grams); mg (milligrams); ° C. (degrees centigrade); L (liters); mL (milliliters); μL (microliters); m (meters); cm (centimeters); mm (millimeters), μm (micrometers); NaOH (sodium hydroxide), H₂SO₄ (sulfuric acid), NaCl (sodium chloride); 11,13-CCLA (9-(6-propyl-cyclohexa-2,4-dienyl)-nonanoic acid), AgNO₃ (silver nitrate).

### EXEMPLE 1

### Preparation of a Mixture Containing High Amounts of 9Z,11E,15Z and 9Z,13E,15Z-C18:3 Acids by Conjugation of Linseed Oil

To commercial propylene glycol (46.48 kg) were added NaOH (1.94 kg) at room temperature. The resulting mixture was heated at 160° C. for 20 minutes into a 200 L stainless steal reactor under a nitrogen atmosphere and with vigorous agitation. Commercial raw linseed oil (4.19 kg) was added under a nitrogen atmosphere. The mixture was heated at 160° C. for 2 hours under a nitrogen atmosphere and with vigorous agitation. After cooling to 80° C., the reaction mixture was directly acidified with an aqueous solution of H₂SO₄ (0.06% w/w, 47.5 kg). After standing for about 10 minutes, the top layer was washed with a NaCl aqueous solution (5% w/w, 47.25 kg). The top layer was removed, dried and stored at −80° C. under nitrogen.

The fatty acid composition of the resulting product was determined using high resolution gas-chromatography following methylation of a sample (20 mg) using boron trifluoride (Metcalfe et al.,). The analytical equipment consisted of an Agilent Technologies GLC 6890 with auto sampler. The column was a highly polar open tubular capillary type. The following program settings were used (TABLE 3)

TABLE 3

| | |
|---|---|
| Inj ction | Split mode 1:50 at 250° C. |
| Det ction | Flame Ionization Detector at 250° C. |
| Carrier | Helium at 249.5 KPa at 170° C. |
| Oven | 60° C. for 1 minute then 20° C./minute to 170° C. and |
| Program | 170° C. throughout for 30 minutes, then 5° C./minute |
| | 210° C. throughout for 5 minutes |
| Column | BPX-70 capillary column, 60 m × 0.25 mm i.d., 0.25 μm film |
| | thickness |

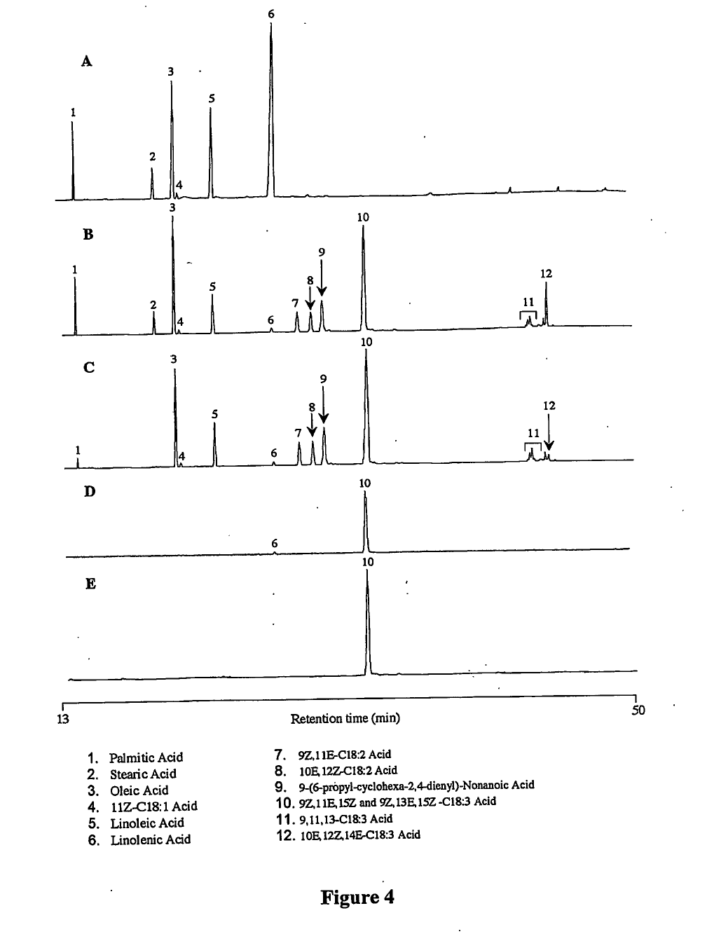

The obtained chromatogram is shown in FIG. 4B. The quantitative conversion of alpha-linolenic acid was confirmed and the mixture comprises approximately 33% of 9Z,11E,15Z and 9Z,13E,15Z-C18:3. The fatty acid composition of the mixture is given in Table 4.

TABLE 4

| Fatty Acid | % Before Reaction | % After Reaction |
|---|---|---|
| Palmitic | 5.40 | 5.07 |
| Stearic | 4.13 | 3.20 |
| Oleic | 19.77 | 19.27 |
| 11Z-C18:1 | 0.69 | 0.65 |
| Linoleic | 16.47 | 7.16 |
| alpha-Linolenic | 53.54 | 0.87 |
| 9Z,11E-C18:2 | 0.00 | 4.89 |
| 10E,12Z-C18:2 | 0.00 | 4.79 |
| 11,13-CCLA | 0.00 | 8.73 |
| 9Z,11E,15Z-C18:3 | 0.00 | 32.98 |
| 9,11,13-C18:3¹ | 0.00 | 3.73 |
| 10E,12Z,14E-C18:3 | 0.00 | 6.06 |
| 10,12,14-C18:3² | 0.00 | 1.41 |

¹stereochemistry of the double bonds not identified
²other stereo isomers of 10,12,14-C18:3 Acid

### EXEMPLE 2

### Preparation of Mixtures Containing High Amounts of a Mixture of 9Z,11E,15Z and 9Z,13E,15Z-C18:3 Acid by Conjugation of Linseed Oil and Consecutive Urea Crystallization

The top layer (3.26 kg) obtained in Example 1 was removed and transferred into a 20 L reactor containing a solution of urea (3.26 kg) in aqueous ethanol (95%, v/v, 13.20 kg), prepared at 60° C. under a nitrogen atmosphere. The free fatty acids were homogenized and the obtained mixture was cooled at 4° C. for 12 h. The liquid phase (17.77 kg) was removed from the solid phase (3.18 kg) by centrifugation and transferred into a 100 L, stainless steal, sight glasses reactor. An aqueous solution of H₂SO₄ (0.1%, w/w, 49.12 kg) was added to the mixture and the solution was vigorously shaken for 1 minute under a nitrogen atmosphere. After standing for 10 minutes, the top layer was washed with an aqueous a NaCl solution (5% w/w, 47.25 kg). The top layer was removed, dried and stored at −80° C. under nitrogen.

The solid phase (3.18 kg) was dissolved in a solution of H₂SO₄ (0.1%, w/w, 49.12 kg) at 70° C. and transferred into a 107 L, stainless steal, sight glasses reactor and the solution was vigorously shaken for 1 minute under a nitrogen atmosphere. After standing for 10 minutes, the top layer was washed in the same apparatus with an aqueous NaCl solution (5% w/w, 47.25 kg). The top layer was removed, dried and stored at −80° C. under nitrogen.

The fatty acid composition of the resulting products was determined using high resolution gas-chromatography following methylation of samples (20 mg) using boron trifluoride (Metcalfe et al.,). The analytical conditions used were the same as presented in Example 1.

The chromatogram obtained is shown in FIG. 4C. The fatty acid composition of the mixture is illustrated in Table 5.

TABLE 5

| | % Before | % in Liquid | % in Solid |
|---|---|---|---|
| Fatty Acid | Crystallization | Phase | Phas |
| Palmitic | 5.07 | 0.58 | 15.41 |
| Stearic | 3.20 | 0.04 | 12.17 |
| Oleic | 19.27 | 17.19 | 27.88 |
| 11Z-C18:1 | 0.65 | 0.66 | 0.84 |
| Linoleic | 7.16 | 8.50 | 2.60 |
| alpha-Linolenic | 0.87 | 0.79 | 0.17 |
| 9Z,11E-C18:2 | 4.89 | 5.86 | 4.17 |
| 10E,12Z-C18:2 | 4.79 | 6.21 | 2.59 |
| 11,13-CCLA | 8.73 | 10.61 | 1.42 |
| 9Z,11E,15Z and 9Z,13E,15Z- | 32.98 | 40.74 | 10.88 |
| C18:3 | | | |
| 9,11,13-C18:3¹ | 3.73 | 3.54 | 3.17 |
| 10E,12Z,14E-C18:3 | 6.06 | 0.73 | 13.78 |
| 10,12,14-C18:3² | 1.41 | 1.26 | 1.72 |

¹stereochemistry of the double bonds not identified
²other stereo isomers of 10,12,14-C18:3 Acid

### EXEMPLE 3

### Preparation and Purification of a Mixture of 9Z,11E,15Z and 9Z,13E,15Z-C18:3 Acids by Reverse Phase Liquid Chromatography

The products obtained in Examples 1 and 2 containing a high level of 9Z,11E,15Z and 9Z,13E,15Z-C18:3 were submitted to a preparative high performance liquid chromatograph fitted with a preparative ODS (octadecylsilyl) column (25 cm×6.5 cm i.d.). The mobile phase was methanol and water (90:10, v/v, 400 mL/minute). The sample (10 g) was injected at atmospheric pressure and the separation was achieved in 60 minutes. The collected fractions were analyzed by gas-liquid chromatography as presented in Example 1, and a typical gas-chromatogram is presented in FIG. 4D. The desired compounds eluted in the first partition (partition number=12) allowing for a purification of about 95%.

### EXEMPLE 4

### Preparation and Purification of 9Z,11E,15Z and 9Z,13E,15Z-C18:3 Acids by Argentation Liquid Chromatography

The fatty acid methyl esters prepared from the products obtained in Examples 1 and 2, containing a high level of a mixture of 9Z,11E,15Z and 9Z,13E,15Z-C18:3, were separated using argentation thin layer chromatography. Silica-gel plates were prepared by immersion in a 5% acetonitrile solution of AgNO₃ as described by Destaillats et al. (Lipids 35:1027-1032, (2000)). The developing solvent was a n-hexane/diethyl ether (90:10, v/v) mixture. At the end of the chromatographic runs, the plates were briefly air-dried, lightly sprayed with a solution of 2′,7′-dichlorofluorescein, and viewed under ultraviolet light (234 nm). The band at R_{f}=0.52 was scraped off and eluted several times with diethyl ether. Complete evaporation of the combined extracts was achieved with a light stream of dry nitrogen. The residues were dissolved in an appropriate volume of n-hexane and analysed by gas-liquid chromatography (purity superior to 98%) as presented in Example 1.

### EXEMPLE 5

### Preparation of Mixture Containing 6Z,8E,12Z,6Z,10E,12Z- and 6Z,9Z,12Z-C18:3 Acids by Partial Conjugation of Borage Oil

NaOH (4.30 g) was added to commercial propylene glycol (96 g) at room temperature. The resulting mixture was heated at 160° C. for 20 minutes under a nitrogen atmosphere and with vigorous agitation. Commercial borage oil (9.35 g) was then added under a nitrogen atmosphere. The mixture was heated at 160° C. for 1 hour under nitrogen and with vigorous agitation. After cooling to 80° C., the reaction mixture was directly acidified with an aqueous solution of H₂SO₄. After standing for 10 minutes, the top layer was washed with a 5% aqueous NaCl solution (w/w, 47.25 kg), removed, dried and stored at −80° C. under nitrogen.

The fatty acid composition of the resulting products was determined using high resolution gas-chromatography after methylation of samples (20 mg) using boron trifluoride (Metcalfe et al.,). The analytical conditions used were the same as presented in Example 1.

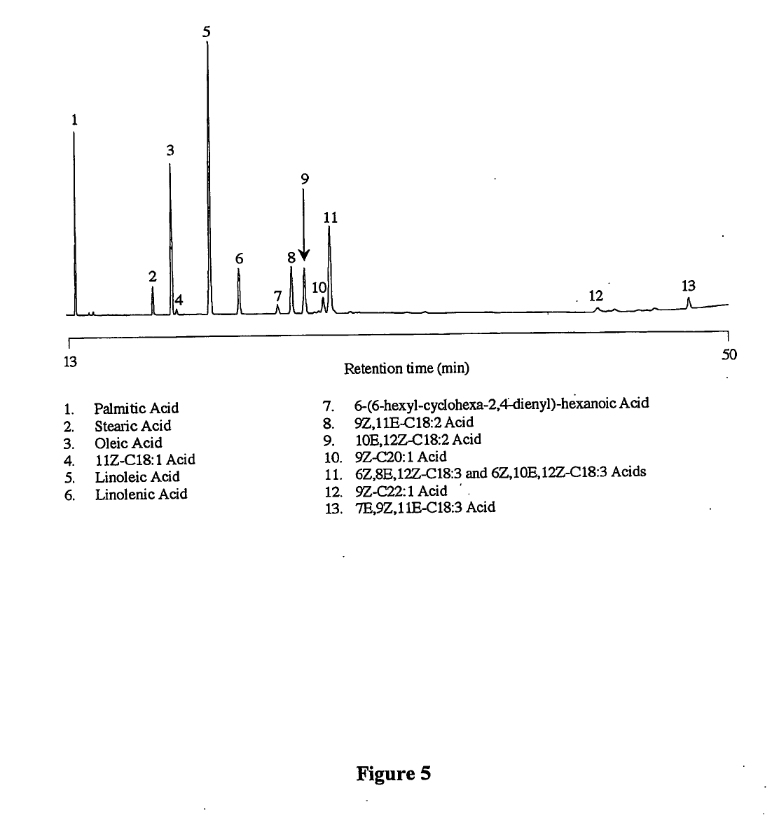

The obtained chromatogram is shown in FIG. 5. The fatty acid composition of the mixture is given in Table 6.

TABLE 6

| Fatty Acid | % Before Reaction | % After Reaction |
|---|---|---|
| Palmitic | 10.34 | 9.55 |
| Stearic | 3.36 | 2.38 |
| Oleic | 15.57 | 13.88 |
| 11Z-C18:1 | 0.57 | 0.52 |
| Linoleic | 39.96 | 30.11 |
| ?-Linolenic | 22.92 | 5.32 |
| 7,11-CCLA | 0.00 | 1.25 |
| 9Z,11E-C18:2 | 0.00 | 6.66 |
| 10E,12Z-C18:2 | 0.00 | 6.46 |
| 9Z-C20:1 | 3.69 | 2.60 |
| 6Z,8E,12Z and 6Z,10E,12Z- | 0.00 | 14.50 |
| C18:3 | | |
| 9Z-C22:1 | 2.05 | 1.22 |
| 7E,9Z,11E-C18:3 | 0.00 | 1.89 |

Although the present invention has been described herein above by way of preferred embodiment thereof, it can be modified without departing from the spirit and nature of the subject invention as defined in the appended claims.

1. A method for preparing conjugated linolenic acids comprising the steps of:
(a) blending a or a mixture of vegetable oils and/or fats including various concentrations of alpha or gamma and or both linolenic acids with a base to produce a reaction mixture;
(b) recovering said conjugated linolenic acids from the reaction mixture; and
(c) subjecting the reaction mixture to urea complexation or liquid chromatography.

2. A method as defined in claim 1, wherein said oils and/or fats are selected from the group consisting of amebia, basil, candelnut, flax (linseed), linola, gold of pleasure, hemp, mustard, perilla, soybean, canola, walnut, chia, crambe, echium, hop, kiwi, pumkin, black currant seed oil, purslane seed oil, borage oil, and evening primrose oil as well as any other oil, wax, ester or amide which comprises free and/or derivatized linolenic acid.

3. A method as defined in claim 2, wherein said base is selected from the group consisting of sodium hydroxide, sodium alkoxylate, sodium metal, potassium hydroxide, potassium alkoxylate, potassium metal and strong base resins.

4. A method as defined in claim 3, further comprising isolating from said reaction mixture geometrical isomers of partially and/or and fully conjugated isomers of said conjugated linolenic acids.

5. A method as defined in claim 1, wherein said blending is performed in a polyol solvent.

6. A method as defined in claim 5, wherein said polyol is selected from the group consisting of propylene glycol, glycerol and ethylene glycol.

7. A method as defined in claim 6, wherein said blending is performed at temperatures ranging from about 20° C. to about 280° C. over a period of time ranging from about 30 seconds to about 18 hours.

8. A method as defined in claim 3, wherein said liquid chromatography is reverse phase liquid chromatography.

9. A method as defined in claim 1, wherein said conjugated linolenic acids are selected from the group consisting of 9Z,11E,15Z-octadecatrienoic acid, 9Z,13E,15Z-octadecatrienoic acid, 6Z,8E,12Z-octadecatrienoic acid, and 6Z,10E,12Z-octadecatrienoic acid.

10. A 6Z,8E,12Z-octadecatrienoic acid of formula 1: 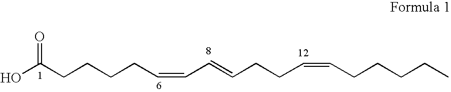
obtained by the method of claim 1.

11. A method for preparing 9Z,11E,15Z-octadecatrienoic acid and 9Z,13E,15Z-octadecatrienoic acid comprising:
(a) blending linseed oil with a base to produce a reaction mixture; and
(b) recovering said conjugated linolenic acids from the reaction mixture.

12. A use of conjugated linolenic acids selected from the group consisting of 9Z,11E,15Z-octadecatrienoic acid, 9Z,13E,15Z-octadecatrienoic acid, 6Z,8E,12Z-octadecatrienoic acid, and 6Z10E,12Z-octadecatrienoic acid in nutritional, cosmetic, and nutraceutical applications, characterized in that the linolenic acids are obtained by the method of claim 1.
